(19) United States
(12) Patent Application Publication
(10) Pub. No.: US 2014/0253684 A1
(43) Pub. Date: Sep. 11, 2014

(54) VISUALIZATION OF REGISTERED SUBSURFACE ANATOMY

(71) Applicants: Rajesh Kumar, Baltimore, MD (US); Russell H. Taylor, Severna Park, MD (US); Thiusius Rajeeth Savarimuthu, Odense (DK); Brian Minnillo, Cleveland, OH (US); Hiep Thieu Nguyen, Boston, MA (US)

(72) Inventors: Rajesh Kumar, Baltimore, MD (US); Russell H. Taylor, Severna Park, MD (US); Thiusius Rajeeth Savarimuthu, Odense (DK); Brian Minnillo, Cleveland, OH (US); Hiep Thieu Nguyen, Boston, MA (US)

(73) Assignee: THE JOHNS HOPKINS UNIVERSITY, Baltimore, MD (US)

(21) Appl. No.: 13/822,135

(22) PCT Filed: May 5, 2011
(86) PCT No.: PCT/US11/35325

§ 371 Date: Oct. 11, 2013

Related U.S. Application Data
(60) Provisional application No. 61/381,749, filed on Sep. 10, 2010.

(51) Int. Cl. H04N 5/33; A61B 1/05

(52) U.S. Cl. CPC H04N 5/33; A61B 1/05
USPC 348/45

(57) ABSTRACT

A system and method for visualization of subsurface anatomy includes obtaining a first image from a first camera and a second image from a second camera or a second channel of the first camera, where the first and second images contain shared anatomical structures. The second camera and the second channel of the first camera are capable of imaging anatomy beneath the surface in ultra-violet, visual, or infra-red spectrum. A data processor is configured for computing registration of the first image to the second image to provide visualization of subsurface anatomy during surgical procedures. A visual interface displays the registered visualization of the first and second images. The system and method are particularly useful for imaging during minimally invasive surgery, such as robotic surgery.

### REFERENCE TO RELATED APPLICATIONS

This application claims the benefit of U.S. Provisional Patent Application No. 61/381,749, filed on Sep. 10, 2010, which is hereby incorporated by reference for all purposes as if fully set forth herein.

### FIELD OF THE INVENTION

The present invention pertains to a system and method for visualization of subsurface anatomy. More particularly, the present invention pertains to a system and method for visualization of subsurface anatomy using two different imaging modalities.

### BACKGROUND OF THE INVENTION

During surgery, it is important that surgeons can adequately visualize the anatomy of a subject. Current operating systems are limited to real-time visual imaging of the subject's anatomy. For example, in laparoscopic surgery, images from a stereo video endoscope are provided to the surgeon, so that the region of interest may be visualized by the surgeon. However, the endoscopic images do not provide any visualization of the subsurface anatomy of the patient.

Similarly, commercial telerobotic surgery systems for soft tissue surgery are generally limited to visual imaging. Telerobotic assistants for minimally invasive surgery have become well established in clinical practice. For example, over 1750 DAVINCI® robotic surgery systems are in clinical use, as well as numerous other robotic camera manipulators such as AESOP™ (Intuitive Surgical, Inc) and ENDOASSIST™ (Prosurgics, Inc.). Robotic surgery is now the dominant treatment for radical prostatectomies being performed in the U.S. Robotic surgery is also in widespread use in the areas of cardiac surgery, and complex gynecological and urological procedures. Robotic surgery is also now being used for partial nephrectomies for the treatment of kidney cancer.

As critical surfaces as well as surgical targets often lie subsurface, a range of visualization techniques have been investigated as robotic surgery gains popularity. This includes visualization of nerves, blood vessels, and tumors. Ultrasound has previously been used to provide registered visualization of tumors in robotic surgery, but ultrasound suffers from noise, poor sensitivity and specificity, and is primarily useful for locating large tumors buried deep below the surface, or guiding instruments to them. Ultrasound also provides a narrow field of view and requires contact manipulation for acquiring any images. Optical coherence tomography (OCT) has also been used for imaging anatomical structures. However, OCT is data intensive, has a small field of view and near-contact imaging, requiring extensive instrumentation and computation, and is therefore unsuitable for imaging large fields of view.

Moreover, when multiple imaging modalities are used, the images are typically displayed as picture in picture images. However, it is difficult to correlate such information with the primary endoscopic view, since it may not relate to the surface visible in the visual endoscopic images. Although picture-in-picture visualizations provide an advantage over a visual endoscopic view alone, it is still difficult for a human to interpret multiple sources of information presented with very different and unrelated viewpoints.

While tools and markers for visualization of nerves, blood vessels, and tumors has seen significant research, integrated imaging of the urinary track has not yet received due attention. In improving situational awareness in urological procedures, mobilization of the ureters is important. Ureteral surgery requires mobilization and transection at ureteropelvic (UPJ) or the ureterovesical junction (UVJ). Mobilization of ureters presents many unique challenges, including disconnecting the ureter from one or more arteries supplying blood to it, leading to ischemia in various degrees, leading to strictures in the anastomosis. The current imaging modalities do not provide any means to effectively image the subsurface ureter during such procedures.

Accordingly, there is a need in the art for an integrated imaging system for real-time multi-modal image registration for visualization of the urinary system. In addition, there is a need in the art to integrate computer vision methods to accurately segment and track anatomical information, such as the ureters and the renal collection system. Finally, there is a need in the art for accurate registration between surface images and subsurface images to create a fused visualization that enhances surgical awareness to make critical uretary tasks easier.

### SUMMARY

According to a first aspect of the present invention, a method for visualization of anatomical structures contained beneath the visible surface comprises obtaining a first image of a region of interest with a first camera, obtaining a second image of the region of interest with a second camera or a second channel of the first camera, the second camera and the second channel of the first camera capable of imaging anatomy beneath the surface in ultra-violet, visual, or infra-red spectrum, the first and second images containing shared anatomical structures, performing a registration between the first and second images, and generating a registered visualization.

According to a second aspect of the present invention, an integrated surgical system for visualization of anatomical structures contained beneath the visible surface comprises a first camera for obtaining a first image of a region of interest, a second camera or a second channel of the first camera for obtaining a second image of the region of interest, the second camera and the second channel of the first camera capable of imaging anatomy beneath the surface in ultra-violet, visual, or infra-red spectrum wherein the first and second images contain shared anatomical structures. A data processor is configured for computing registration of the first camera to the second camera or second channel of the first camera, and a visual interface is positioned to display the registered visualization.

### BRIEF DESCRIPTION OF THE DRAWINGS

The accompanying drawings provide visual representations which will be used to more fully describe the representative embodiments disclosed herein and can be used by those skilled in the art to better understand them and their inherent advantages. In these drawings, like reference numerals identify corresponding elements and:

FIG. 1 is a schematic of an exemplary imaging system according to features of the present invention.

FIG. 2 is a schematic of an exemplary method according to features of the present invention.

FIG. 3 is a schematic of an exemplary method according to features of the present invention.

FIG. 4 is a schematic of an exemplary method according to features of the present invention.

FIG. 5 is a photograph of a registered overlay of a near infrared image onto a stereo endoscopic image according to features of the present invention.

### DETAILED DESCRIPTION OF THE PREFERRED EMBODIMENTS

The presently disclosed subject matter now will be described more fully hereinafter with reference to the accompanying Drawings, in which some, but not all embodiments of the inventions are shown. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

The present invention pertains to a system and method for visualization of subsurface anatomy during any type of surgical procedure, including but not limited to, laparoscopic surgery, robotic surgery, and other minimally invasive surgeries, as well as open surgery. The present invention allows for imaging from two sources of a region of interest. In the following embodiment, a two source example is given. However, the invention can utilize more than two sources of images. The first source obtains a first image of the region of interest, and the second source obtains a second image of the region of interest with a second camera or second channel of the first camera capable of imaging anatomy beneath the surface, wherein the first and second images contain shared anatomical structures. Registration is performed between the first and second images so that a registered visualization may be generated. While the exemplary embodiment of the present invention is primarily described in the context of a robotic surgical system, it should be understood that the system and method of the present invention are applicable to other surgical platforms, such as freehand laparoscopic surgery and other minimally invasive surgeries, as well as open surgery.

With reference to FIG. 1, the system and method of the present invention is described in connection with an exemplary robotic surgical system 2. One example of a robotic surgical system which may incorporate the method and system of visualization of the present invention is a DAVINCI® system, manufactured by Intuitive Surgical, Inc. of Mountain View, Calif. As is known in the art, a robotic surgical system 2 includes a master control station 4 including a surgeon's console. The surgeon's console preferably includes a pair of master manipulators and a display, which allow the surgeon to view 3-dimensional auto stereoscopic images and manipulate one or more slave stations. In addition to 3-dimensional auto stereoscopic imaging, the display also allows for simultaneous visualization of multiple video sources.

The robotic surgical system 2 may include any number of slave stations, including but not limited to, a vision cart 6 for housing the stereo endoscopic vision and computing equipment, and a patient cart 8 with one or more patient side manipulators 10. As is known in the art, a wide range of easily removable surgical instruments may be attached to the patient side manipulators 10 of the patient cart 8, which move in response to the motion of the master manipulators at the surgeon's console. In addition, it should be understood that a robotic surgical system according to features of the present invention may include one or more master manipulators, as well as any number of slave manipulators, as is known in the art.

When performing surgery, the system and method of the present invention allows for more comprehensive visualization of subsurface anatomy. In the context of robotic surgery, the first camera 12 may be attached to a patient side manipulator of the patient cart 8. Preferably, the first camera 12 is stereo endoscopic camera capable of imaging the surface of a region of interest. However, it should be understood that the first camera 12 may be any type of camera capable of imaging the surface of the region of interest. In the context of robotic surgery, the images acquired by the endoscope 12 may be displayed on the auto stereoscopic display of the surgeon's console, to thereby direct the surgeon during surgery. The images may also be directed to the vision cart 6, to allow for display thereon.

In the context of laparoscopic surgery, a first camera 12 may be positioned within a port while the second camera 20 may be positioned within another port. The first camera 12 obtains a first image, and the second camera 20 obtains a second image, wherein each the first and second images contain shared anatomical structures. The second camera 20 is capable of imaging anatomy beneath the surface in ultra-violet, visual, or infra-red spectrum. A registration is performed between the first and second images, which generates a registered visualization. Similarly, in open surgery, the first camera 12 and second camera 20 should be positioned such that the first and second images obtained from the first camera 12 and second camera 20 contain shared anatomical structures. The images are then processed and registered to generate the registered visualization.

However, it should that the first camera may include two channels in which to view different types of images. In this way, a first channel would be operable to obtain the first image of the region of interest and the second channel of the first camera would be capable of imaging anatomy beneath the surface in ultra-violet, visual, or infra-red spectrum to obtain the second image of the region of interest.

According to features of the present invention, the images acquired by the first camera 12 must be further processed to enable the registered visualization according to features of the present invention. In particular, the images obtained from the first camera 12 are preferably sent to a work station 14. The workstation 14 includes a data processor 16 or computer system for performing the visualization according to features of the present invention. The data processor includes a memory device 18 having a program with machine readable instructions for performing the necessary algorithms for generating the visualization according to features of the present invention. The workstation 14 may be a stand-alone computer system, or could be incorporated into existing software. For example, in the context of robotic surgery, the data processor 14 could be incorporated into existing software for the DAVINCI®surgical systems.

In addition to traditional images generated from an endoscopic camera and the like, a camera-like, second camera 20 (or the second channel of the first camera) is provided for imaging of subsurface anatomy. Preferably, the second camera 20 (or the second channel of the first camera) is capable of imaging anatomy beneath the surface in the ultra-violet, visual, and infra-red spectrum. In the exemplary embodiment, the second camera or the second channel of the first camera is a near infrared imager (NIR). The NIR images provide anatomical features (e.g., the ureters and collecting system) located slightly beneath the surface, from a different view. Near infrared (NIR) fluorescent imaging may capture other relevant anatomy not visible in the endoscopic visible light imaging. Fluorescence occurs when a fluorophore decays and emits a NIR photon which then can be sampled and visualized. NIR imaging has been used to visualize the urinary track for characterizing of metabolism in the urine, as well as detection of bladder cancer. However, other types of cameras may be used, such as an IR (infrared) imager, far infrared imager (FIR), and the like.

Like the images from the first camera 12, the images from the second camera 20 (or the second channel of the first camera) are preferably sent to the workstation 14, and processed therein. As described above, the memory device 18 includes machine readable instructions for performing the necessary algorithms for generating the visualization according to features of the present invention.

For the DAVINCI® robotic surgical system, streaming measurements of the motion of its manipulators is possible. In particular, the Application Programming Interface (API) provides transparent access to motion vectors including joint angles and velocities, Cartesian position and velocities, gripper angle, and joint torque data. The DAVINCI® robotic surgical system may also include the current stereo endoscopic camera pose, and changes in the camera pose. The API can be configured to stream at various rates (up to 100 Hz) for providing manipulation data better than video acquisition rates. The API provides data useful for registration of the endoscopic images to the subsurface images.

As summarized in FIG. 1, the images from the first camera 12 and the second camera 20 (or the second channel of the first camera) preferably go through the following steps: Image Acquisition, Segmentation and Preprocessing, Registration, and Visualization and Interaction, which will be described in more detail below. Prior to image acquisition, the first camera 12 and second camera 20 (or the second channel of the first camera) are preferably calibrated to identify intrinsic and extrinsic camera parameters. Calibration is a simple process and may be repeated whenever there is a reconfiguration of the optical chain. For example, the cameras may be calibrated using the Camera Calibration Toolbox for MATLAB.

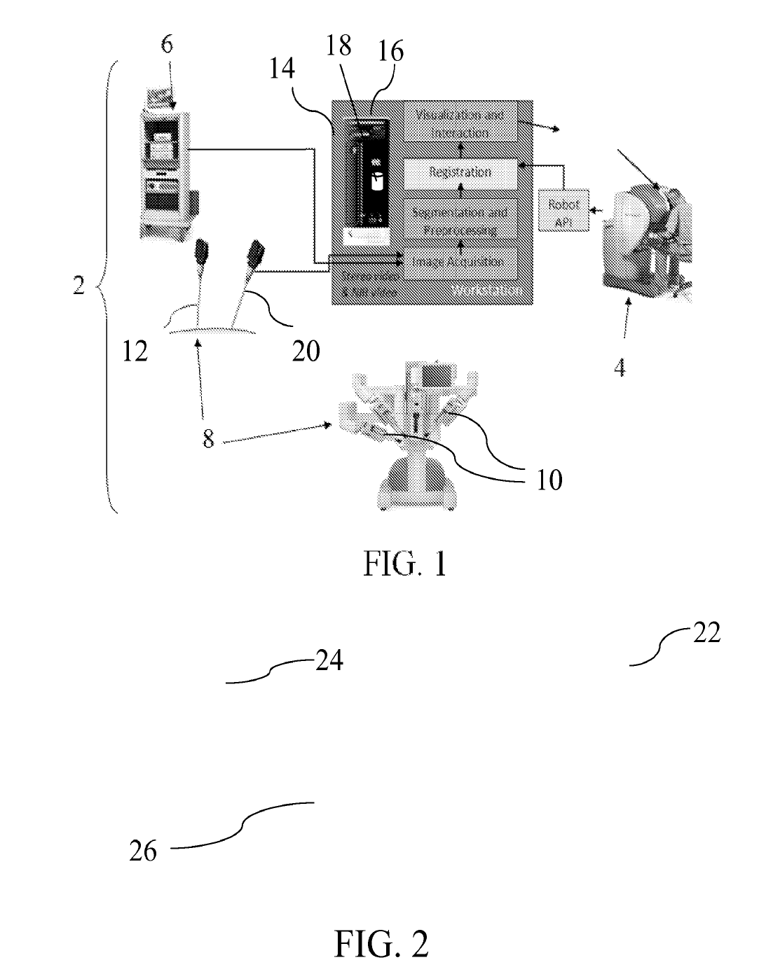

Once the cameras are calibrated, the images are acquired from the first camera 12 and the second camera 20 (or second channel of the first camera). With reference to FIG. 2, a first image 22 of a region of interest is obtained with the first camera and a second image 24 of the region of interest is obtained with the second camera (or second channel of the first camera). The first image 22 is shown as a stereo image taken from an endoscope and the second image 24 is shown as a mono image taken from an IR camera. However, it should be understood that the first image 22 may be either a stereo or mono image, and the second image 24 may be either a stereo or mono image. Moreover, other types of images are possible, and within the scope of the present invention.

Once the first and second images are acquired, the images are processed so that they may be registered to one another. According to features of the exemplary embodiment, the first image 22 and the second image 24 are rectified using previously computed calibration parameters. Corresponding features in the rectified images are then used to find the 3-dimensional position of each fiducial point in respective image spaces, i.e., 3-dimensional points in the endoscope view and 3-dimensional or 2-dimensional positions in the subsurface view.

Once the correspondences are established between the 3-dimensional positions in the stereo images and image features in the NIR imager, the homogeneous equation AX=XB is solved to obtain a registration transformation T=(R, p) between the registration fiducials and the respective imager. Given the two transformations Tᵢ=(Rᵢ, pᵢ), the registration between the two imagers is obtained by appropriate composition Tᵢⱼ=(RᵢRⱼ^{T}, pᵢ−R_{i,}Rⱼ^{T}pⱼ) of the individual transformations. The registration then allows for an overlay, picture-in-picture visualization or fusion of the image to be created. Although a rigid registration between image plane is described here, the method is equally applicable with non-rigid 2D-2D, 2D-3D and 3D-3D registration methods employing surfaces and volumes extracted from the camera images (or associated preoperative CT/MR image data). In such a case separate registrations will be performed between the first camera and the second camera visualizing the subsurface anatomy, and the second camera and the preoperative imagery. This will establish a registration between the three spaces that can be updated in real-time without any contact-based/intrusive/radiation imaging. Interactive, landmark based, and automated registration methods all apply equally towards establishing the feature points for such a registration.

After the registration method is performed, the second image 24 is overlaid on top of the first image 22, thereby creating a fused overlay 26. This creates a visualization of the subsurface anatomy which is not possible with the endoscope alone. That is, the registered visualization fuses the first and second images to create a single view. The overlay provides important information regarding the structure of the anatomy which is not visible from the surface images obtained by the endoscope. While an overlay 26 is shown, a picture-in-picture visualization, and the like, is possible and within the scope of the invention.

Preferably, registration is performed in real time and is updated when there is a change in position of the first camera or second camera. However, it should be understood that registration with the collected images may be maintained after one camera is removed, or if the camera is no longer producing good images, due to the fluorescing marker being excreted. This is accomplished by relying upon previous images stored in the data processor, and not on real-time images from the nonfunctioning camera.

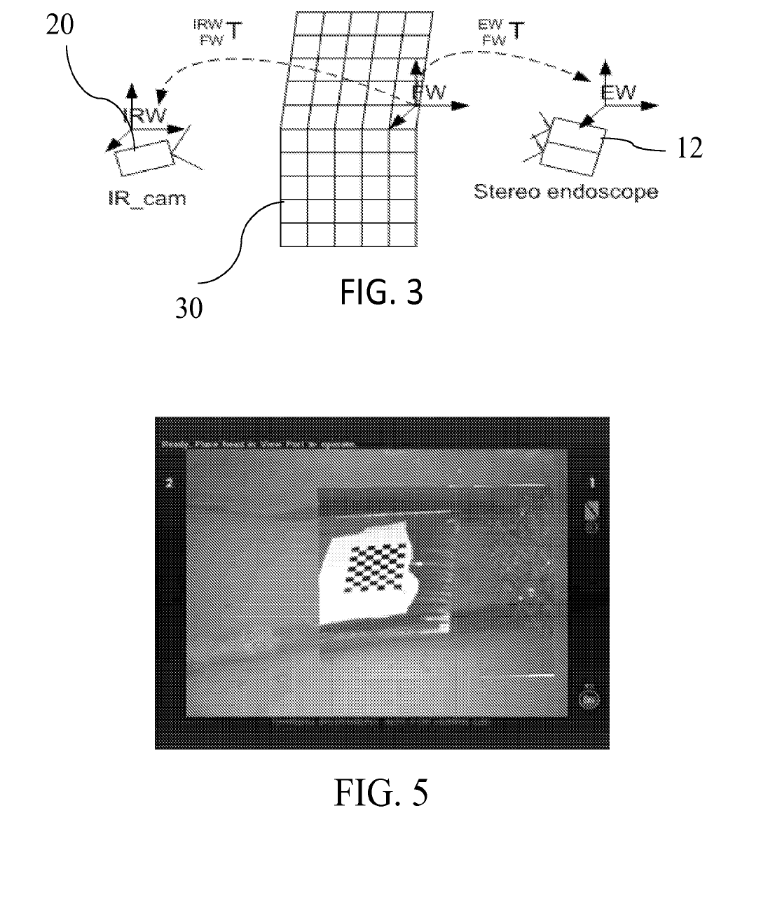

With reference to FIG. 3, details of an exemplary registration method using stereo images from the first camera and second camera (or second channel of the first camera) are illustrated. In particular, a feature based registration method is illustrated, which involves the extraction of corresponding features of each image to be registered. These features include, but are not limited to, color, edge, corner, texture, or more robust features, which are then used to compute the transformation between the two images. Preferably, features are chosen that are robust to changes in illumination and are available in both the first camera and second camera imaging range to match the dynamic surgical environment. Such features include spatially and kernel weighted features as well as gradient features located in anatomical landmarks. Detected features may be tracked using standard methods such as sum of squared distances (SSD) approach. In rectified stereo pairs, feature correspondences may then be computed using image similarity measures, such as normalized cross-correlation (NCC), sum of squared differences (SSD), or zero-mean SSD. A mapping (disparity map) between the image coordinates for the features in the stereo pair(s) is then formulated as an over-constrained linear system and solved. However, while a featured based registration method is primarily described in connection with the exemplary embodiment of the present invention, it should be understood that any type of registration is possible, including area based registrations, as more fully described by Zitova et al., “Image registration methods: a survey”, Image and Vision Computing, 21(11): 977-1000 (2003), the entire disclosure of which is incorporated by reference herein.

In addition, it should also be understood that the registration method may compute a single rigid homogeneous transform or a deformable map aligning the two reconstructed surfaces from the two image sources. When applying a rigid registration, registration is between image planes of the first and second images. When the first image is a stereo image and the second image is a stereo image, the registration may be by way of planar geometry. When applying deformable registration, a relationship between registered 2D-3D or 3D-3D points allows for deformation of the subsurface image for visualization. Accordingly, deformable registration may be performed between representations created from stereo images. As is known in the art, deformable registration may use surfaces, volumes, and the like.

According to the exemplary embodiment, points on a fiducial marker 30 in the region of interest are used to register the two images. During surgery, the fiducial marker 30 may be an object placed onto the subsurface anatomy, as is known in the art. In addition, the fiducial marker 30 may be virtual, for example, by using a structured light system. Further, the fiducial marker may be anatomical landmarks. In this regard, the anatomical landmarks may be annotated or marked interactively. Alternatively, only some of the anatomical landmarks may be annotated or marked interactively, while the remaining registration is performed automatically (e.g. using methods such as SIFT, or SURF). Still further, registration may be completely automatic, using methods such as SIFT or SURF.

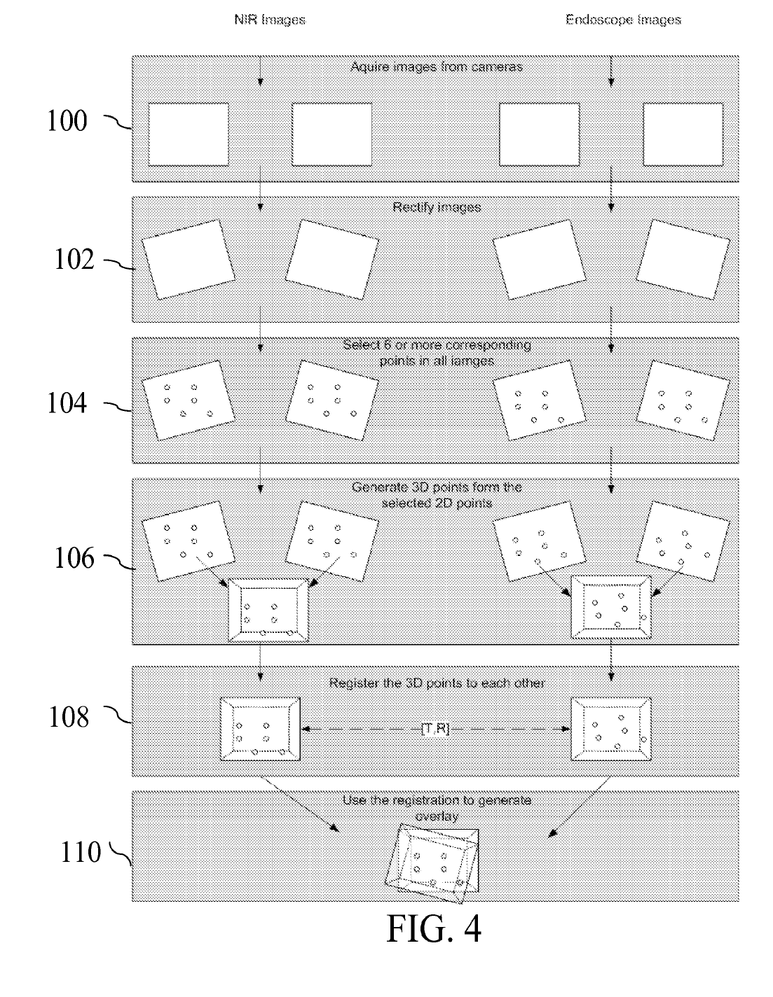

With reference to FIG. 4, an exemplary registration method according to features of the present invention is illustrated. The registration method features an endoscope (first camera) and an NIR imager (second camera or second channel of the first camera). However, as described above, numerous other imaging modalities may be used for the first camera and the second camera or second channel of the first camera. At step 100, stereo images are acquired from the endoscope and the NIR imager. In step 102, each image pair is rectified using previously computer calibration parameters. At step 104, corresponding feature points are located in each pair. According to the exemplary method, at least six feature points are detected. However, fewer or greater feature points may be selected according to application and design preference.

At step 106, 3-dimensional points for the endoscopic images are preferably generated of the selected feature points using camera parameters and 3-dimensional points for the subsurface images are generated of the selected feature points of the subsurface image. However, as described above, it should be understood that the subsurface image may be a mono image, which can be used to generate a 2-dimensional point for the subsurface image.

At step 108, the selected feature points of said endoscope image is registered to the selected feature points of the NIR image using the registration transformation described above. At step 110, the registration is used to generate an overlay or picture-in-picture visualization of the two images, which can then be updated with any motion. The visualizations are then displayed on a visual interface.

In the context of robotic surgery, the visualizations are preferably displayed on the surgeon's console, or a display on the vision cart 6 or patient cart 8 (FIG. 1). In the context of laparascopic surgery, the visual interface may be a display positioned adjacent the surgeon. In this way, the visualization is used as an intra-operative display. In addition, the visualization may generate separate registered images (picture-in-picture visualizations) and the visual interface may be a multi-view display. However, any numerous types of displays and registrations are possible, depending upon application and design preference.

In addition, the surgeon may further manipulate the images in a “masters as mice” mode, where the master manipulators are decoupled from the slave manipulators and used as 3D input devices to manipulate graphical objects in the 3D environment. For example, the surgeon can move the overlay to a different region of the visual field so that it does not obstruct the view of important anatomy. See, for example, U.S. Patent Publication No. 2009/0036902, the entire content of which is incorporated by reference herein.

Accordingly, the present invention provides an integrated surgical system and method that allows for registered visualizations of the subsurface anatomy of a patient from two separate imaging sources, so that the subsurface anatomy of a patient is more accurately visualized during surgical procedures. This technology will be a great benefit for intricacies of ureter mobilization, and as well as other highly sensitive operations.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The following Examples are offered by way of illustration and not by way of limitation.

### Example 1

A nontoxic ballistic gel phantom containing simulated bladder and ureters and non-clinical chemi-luminescent agent appropriate for both NIR and stereo endoscopic imaging was used for engineering validation with the DAVINCI S® robotic surgery system. In a first experiment, the phantom and NIR imager were placed in a torso model with endoscopic ports to collect mono and stereo NIR video, and stereo endoscopic video. A custom stereo infrared imager prototype was constructed having two cameras supplied by Videre Design, located in Menlo Park, Calif.

With the prototype imagers, usable registration accuracy was obtained (less than 6 pixels in the stereo image space) using as few as 6 features. This average RMS error falls below 3 pixels (maximum 4.93 pixels) with the use of 14 feature points. Table I contains representative fiducial registration errors.

TABLE 1
FIDUCIAL REGISTRATION ERRORS USING 14 FIDUCIALS

| Fiducial | RMS Error |
|---|---|
| 1 | 2.98 |
| 2 | 2.51 |
| 3 | 1.94 |
| 4 | 3.40 |
| 5 | 0.76 |
| 6 | 1.64 |
| 7 | 1.27 |
| 8 | 1.45 |
| 9 | 3.76 |
| 10 | 1.68 |
| 11 | 3.95 |
| 12 | 3.75 |
| 13 | 3.49 |
| 14 | 4.93 |
| Average | 2.67 |

### Example 2

An initial pre-clinical experiment was performed on a 30-40 kg female swine model which had been injected with Genhance-750, 1.5 mg/kg via the ear vein, having short looped nephrons and urine transport characteristics similar to the human kidney. NIR Imaging was performed using a prototype photodynamic eye (Hamamatsu PDE), together with acquisition of DAVINCI™ stereo endoscopic video. Registration was performed with 14 feature points with an average RMS error of 2.67 pixels. FIG. 5 shows the registered image overlay of the NIR image on the endoscopic image. As shown in FIG. 5, the subsurface ureters are more dramatically visible in the overlaid picture, enhancing surgical awareness and making critical uretary tasks such as mobilization of the ureters easier.

Although the present invention has been described in connection with preferred embodiments thereof, it will be appreciated by those skilled in the art that additions, deletions, modifications, and substitutions not specifically described may be made without departing from the spirit and scope of the invention as defined in the appended claims.

1. A method for visualization of anatomical structures contained beneath the visible surface, comprising:
obtaining a first image of a region of interest with a first camera;
obtaining a second image of the region of interest with a second camera or a second channel of the first camera, said second camera and said second channel of the first camera both being capable of imaging anatomy beneath the surface in ultra-violet, visual, or infra-red spectrum;
performing a registration between said first and second images; and
generating a registered visualization.

2. The method of claim 1, wherein the registered visualization fuses said first and second images to create a single view.

3. The method of claim 1, wherein the registered visualization generates separate registered images in multi-view displays.

4. The method of claim 3 wherein the multi-view display is a picture-in-picture visualization.

5. The method of claim 1, wherein the registration is performed using anatomical landmarks.

6.-27. (canceled)

28. An integrated surgical system for visualization of anatomical structures contained beneath the visible surface, comprising:
a first camera positioned to obtain a first image of a region of interest;
a second camera or a second channel of the first camera positioned to obtain a second image of the region of interest, said second camera and the second channel of the first camera both being capable of imaging anatomy beneath the surface in ultra-violet, visual, or infra-red spectrum, said first and second images containing shared anatomical structures;
a data processor configured for computing registration of the first camera to the second camera or second channel of the first camera; and
a visual interface positioned to display a registered visualization.

29. The system of claim 28, wherein the registered visualization includes a fusion of said first and second images to create a single view.

30. The system of claim 28, wherein the registered visualization includes separate registered images and the visual interface is a multi-view display.

31. (canceled)

32. The system of claim 28, wherein the registration is performed using anatomical landmarks.

33. (canceled)

34. (canceled)

35. The system of claim 28, wherein the registration is performed using image features.

36. (canceled)

37. (canceled)

38. The system of claim 35, wherein 3-dimensional points are generated for said first image from selected ones of said image features and 3-dimensional points are generated for said second image from selected ones of said image features of said second image prior to registration.

39. The system of claim 35, wherein said image features are taken from a fiducial marker in the region of interest.

40. (canceled)

41. (canceled)

42. The system of claim 39, wherein said fiducial marker is an anatomical landmark of the anatomical structures contained beneath the visible surface of the subject.

43. The system of claim 28, wherein the registration is rigid registration between image planes of said first and second images.

44. The system of claim 28, wherein if the first image is a stereo image and the second image is a stereo image, then the registration is by way of planar geometry.

45. The system of claim 28, wherein a deformable registration is performed between representations created from stereo images.

46. (canceled)

47. (canceled)

48. The system of claim 28, wherein the registration is updated when there is a change in position of the first camera or second camera.

49. The system of claim 28, wherein the first camera is a stereo video camera, and wherein the second camera or second channel of the first camera is a near infra-red imager.

50.-54. (canceled)

55. The system of claim 28, further comprising a surgical robot.

56. The system of claim 55, further comprising a robotic apparatus for manipulating the first camera and the second camera.
